(19) United States
(12) Patent Application Publication
(10) Pub. No.: US 2009/0247494 A1
(43) Pub. Date: Oct. 1, 2009

(54) Omega-3 essential fatty acid composition and method for treatment of anal and colon-rectal disorders

(76) Inventor: Phillip Mark Kofsky, Ambler, PA (US)

Correspondence Address: Phillip Kofsky, 713 Lake Drive, Ambler, PA, 19002, US

(21) Appl. No.: 12/414,414

(22) Filed: Mar. 30, 2009

Related U.S. Application Data
(60) Provisional application No. 61/041,410, filed on Apr. 1, 2008.

(51) Int. Cl. A61K 31/56

(52) U.S. Cl. 514/171

(57) ABSTRACT

I have developed a unique pharmaceutical composition containing Omega-3 essential fatty acids and advantageously an optional corticosteroid and/or topical anesthetic. The composition is useful in a method for treating anal disorders such as fissures and ulcers, hemorrhoids, pruritis ani, and colon-rectal disorders such as procto-sigmoiditis by topical application to or proximate the affected area.

This application emanates from a previously filed provisional application No. 61/041,410 filed Apr. 1, 2008

### FIELD OF THE INVENTION

The present invention relates generally to both an internal and external medical treatment and method. More particularly, the present invention relates an omega-3 essential fatty acid composition treatment for anal and colon-rectal disorders.

### BACKGROUND OF THE INVENTION

Hemorrhoids are the result of swollen, normally present blood vessels in and around the anus and lower rectum. When these normally present veins become abnormally enlarged or dilated, they are called hemorrhoids (also known as hemorrhoids or piles). In this respect, they have been compared to varicose veins in the legs. Hemorrhoids may be either inside the anus (internal) or under the skin around the anus (external).

Hemorrhoids are extremely common in both the male and female populations with it being reported that the majority of people will have experience of or problems from hemorrhoids during their life. Symptoms of hemorrhoids include anal bleeding while some external hemorrhoids may cause painful swelling or a hard lump. Hemorrhoids are usually not dangerous or life threatening although it is important that they be evaluated to exclude more serious causes of the symptoms such as polyps and cancer.

A great deal of the medical treatment of hemorrhoids is aimed at relieving the symptoms and these can include warm tub or sitz baths, ice packs to reduce swelling and application of a hemorrhoidal cream or suppository to the affected area for a limited time. This may be accompanied by changes to the diet and it is often recommended to increase the amount of fiber and fluids in the diet to result in softer bulkier stools to eliminate the pressure on the hemorrhoids.

Surgical treatment includes rubber band ligation to cut off circulation to the hemorrhoid whereby it withers away within a few days. Sclerotherapy comprises the injection of a chemical solution around the blood vessel to shrink the hemorrhoid. Other techniques used may include electrical or laser heat or in the case of more extensive or severe hemorrhoids, an operation called an hemorrhoidectomy may be performed.

As aforementioned, there are certain creams and suppositories which are known in the art to relieve the symptoms of hemorrhoids and other colon-rectal disorders.

As a colon-rectal surgeon, there is an unmet need for an improved product to treat patients with ano-rectal conditions.

### SUMMARY OF THE INVENTION

The present invention relates to an omega-3 essential fatty acid composition and method for treatment of anal and colon-rectal disorders.

The product is comprised of the following components:

EPA and DHA
EPA essential fatty acid-anti-inflammatory
DHA essential fatty acid-anti-inflammatory
Hydrocortisone 1%-anti-itch, anti-inflammatory
Pramoxine HCl 1%-local anesthetic
Lidocaine 1%-local anesthetic
For external direct application, pramoxine HCl or lidocaine is added as a local anesthetic.
Also, the product will be available with and without hydrocortisone to relieve itching and inflammation.
It is available in a cream, ointment, gel or lotion for external direct application and in suppository and enema form for internal application.
   Components working together—many formulations to be available: illustrative examples
Suppository-EPA 400 mg, DHA 250 mg in a selected carrier
Suppository-EPA 400 mg, DHA 250 mg, Hydrocortisone 1% in a selected carrier
Enema-EPA 9.6 grams, DHA 6 grams in a selected carrier
Cream-EPA 800 mg, DHA 500 mg, Lidocaine 1% in a selected carrier
Cream-EPA 800 mg, DHA 500 mg, Pramoxine HCl 1% in a selected carrier
Cream-EPA 800 mg, DHA 500 mg, Lidocaine 1%, Hydrocortisone 1% in a selected carrier
Cream-EPA 800 mg, DHA 500 mg, Pramoxine HCl 1%, Hydrocortisone 1% in a selected carrier

A unique pharmaceutical composition available in a cream, ointment, gel or lotion for external direct application to the anal area and in suppository and enema form for internal rectal application.

1. I have developed a unique pharmaceutical composition containing Omega-3 essential fatty acids and advantageously an optional corticosteroid and/or topical anesthetic. The composition is useful in a method for treating anal disorders such as fissures and ulcers, hemorrhoids, pruritis ani, and colon-rectal disorders such as procto-sigmoiditis by topical application to or proximate the affected area. Symptomatic lo hemorrhoids are inflamed and swollen blood vessels around the anus. Fissures are tears or ulcers around the anus. Pruritis ani is severe itching around the anus. Procto-sigmoiditis is inflammation of the lining of the lower colon and rectum. The unique and key ingredient to my formulation is Omega-3 essential fatty acids which have proven to be anti-inflammatory in human tissue and cells. They are specifically EPA (eicosapentaenoic acid) and DHA (docosahexaenoic acid). These essential fatty acids are not produced in the human body and must be obtained from outside sources such as fish oil. Essential fatty acids are an essential building block for human cell function. My unique product does not require a doctor's prescription and it has been formulated by a colon and rectal specialist. It is applied directly to the anal area or internally into the rectum. The product relieves the following symptoms: swelling, burning, itching, pain, irritation and inflammation. These symptoms can arise from sitting, standing, constipation, diarrhea, lifting, coughing, obesity, pregnancy and childbirth. Less often, these symptoms can arise from colitis, radiation treatment to this area and chemotherapy treatment for malignancies. It is available in a cream, ointment, gel or lotion for external direct application and in suppository and enema form for internal application. For external direct application, pramoxine HCl or lidocaine is added as a local anesthetic. Also, the product will be available with and without hydrocortisone to relieve itching and inflammation. The composition further comprises a carrier selected from the group consisting of white petrolatum, mineral oil, lanolin, distilled water, acetone, cocoa butter and paraffin.

2. An Omega-3 essential fatty acid composition and method for treatment of anal and colon-rectal disorders comprising;
a. EPA essential fatty acid-anti-inflammatory
b. DHA essential fatty acid-anti-inflammatory
c. Hydrocortisone 1%-anti-itch, anti-inflammatory
d. Pramoxine HCl 1%-local anesthetic
e. Lidocaine 1%-local anesthetic

3. The treatment of claim 2 wherein hydrocortisone may be present.

4. The treatment of claim 2 wherein the treatment is formulated as a cream, ointment, gel, lotion, enema or suppository form.
